(19) United States
(12) Patent Application Publication
(10) Pub. No.: US 2024/0032840 A1
(43) Pub. Date: Feb. 1, 2024

(54) MYOELECTRIC-SENSOR MOUNTING MEMBER AND MYOELECTRIC MEASUREMENT DEVICE

(71) Applicant: Alps Alpine Co., Ltd., Tokyo (JP)

(72) Inventors: Mayu Endo, Tokyo (JP); Satoshi Shindo, Miyagi-ken (JP); Takayuki Ito, Miyagi-ken (JP)

(73) Assignee: Alps Alpine Co., Ltd., Tokyo (JP)

(21) Appl. No.: 18/481,423

(22) Filed: Oct. 5, 2023

Related U.S. Application Data
(63) Continuation of application No. PCT/JP2022/010137, filed on Mar. 8, 2022.

(30) Foreign Application Priority Data
Apr. 22, 2021 (JP) 2021-072626

(51) Int. Cl. A61B 5/296; A61B 5/256; A61B 5/257; A61B 5/313; A61B 5/00

(52) U.S. Cl. CPC A61B 5/296; A61B 5/256; A61B 5/257; A61B 5/313; A61B 5/7221; A61B 2562/225; A61B 2562/164

(57) ABSTRACT

A myoelectric sensor mounting member includes a mounting portion that is mounted on a measurement site of a living body, and a holding portion that is capable of holding a myoelectric sensor at an optional rotational angle.

### CLAIM OF PRIORITY

This application is a Continuation of International Application No. PCT/JP2022/010137 filed on Mar. 8, 2022, which claims benefit of and priority to Japanese Patent Application No. 2021-072626 filed on Apr. 22, 2021. The entire contents of each application noted above are hereby incorporated by reference.

### BACKGROUND

### 1. Field

The present disclosure relates to a myoelectric sensor mounting member and a myoelectric measurement device.

### 2. Description of the Related Art

Technology to output myoelectric signals of a living body by a myoelectric sensor is known (e.g., see Japanese Unexamined Patent Application Publication No. 2018-114262).

However, conventional myoelectric sensors are limited with regard to sites of the living body that are the object of measurement, and have dedicated mounting portions for such sites that are the object of measurement. Accordingly, myoelectric signals cannot be measured at other sites of the living body with high precision

### SUMMARY

An embodiment of the present disclosure provides a myoelectric sensor mounting member including a mounting portion for mounting to a measurement site of a living body, and a holding portion capable of holding a myoelectric sensor at an optional rotational angle, the myoelectric sensor comprising a detection electrode on a contact face for contacting the measurement site, in which an orientation of the detection electrode as to the measurement site can be changed by changing the rotational angle at which the holding portion holds the myoelectric sensor.

According to an embodiment, myoelectric signals at a wide variety of measurement sites of the living body can be detected with higher precision.

### BRIEF DESCRIPTION OF THE DRAWINGS

FIG. 1 is an external perspective view of a myoelectric measurement device according to a first embodiment;

FIG. 2 is a plan view of a myoelectric sensor mounting member that the myoelectric measurement device according to the first embodiment includes;

FIG. 3 is a block diagram illustrating a functional configuration of a control unit that a myoelectric sensor according to the first embodiment includes;

FIG. 4 is a diagram showing an example of measurement data measured by the myoelectric sensor according to the first embodiment;

FIG. 5 is a diagram showing an example of measurement data measured by the myoelectric sensor according to the first embodiment;

FIG. 6 is a diagram showing an example of measurement data measured by the myoelectric sensor according to the first embodiment;

FIG. 7 is a diagram illustrating a mounting example of the myoelectric sensor according to the first embodiment, using a belt;

FIG. 8 is an external perspective view of a myoelectric measurement device according to a second embodiment;

FIG. 9 is an external perspective view of the myoelectric measurement device according to the second embodiment;

FIG. 10 is an exploded perspective view of the myoelectric measurement device according to the second embodiment;

FIG. 11 is an exploded perspective view of the myoelectric measurement device according to the second embodiment;

FIG. 12 is an external perspective view illustrating a modification of the myoelectric measurement device according to the first embodiment; and

FIG. 13 is a plan view illustrating a modification of the myoelectric sensor mounting member that the myoelectric measurement device according to the first embodiment includes.

### DESCRIPTION OF THE EXEMPLARY EMBODIMENTS

An embodiment will be described below with reference to the drawings.

### First Embodiment

### Configuration of Myoelectric Measurement Device 100

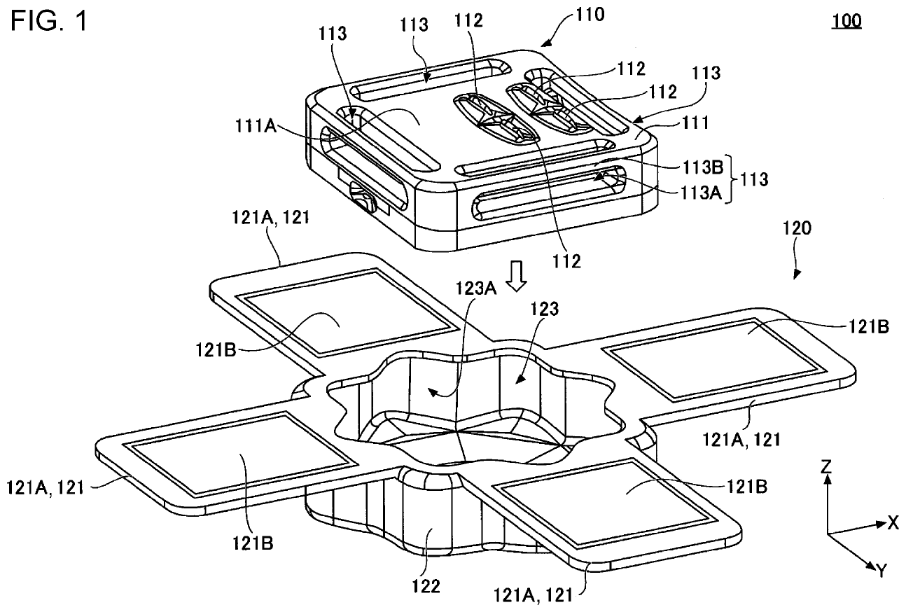

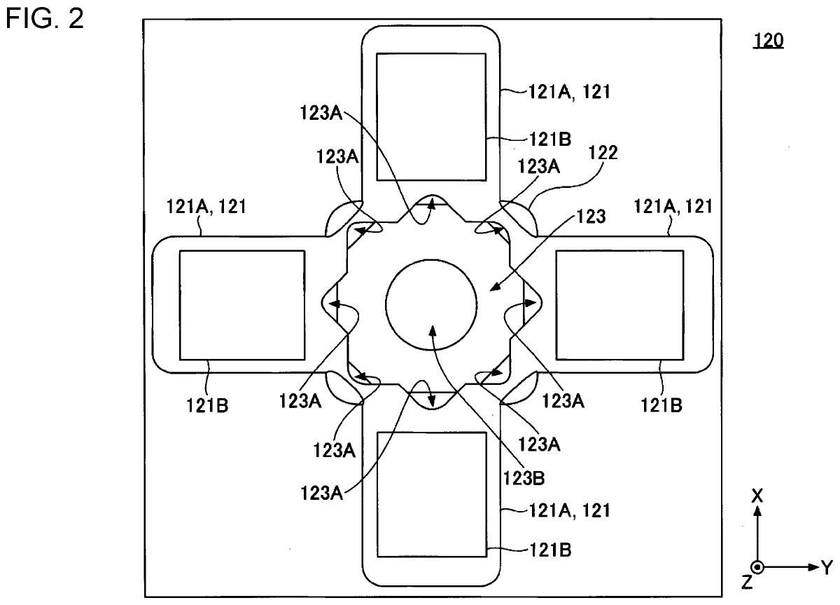

FIG. 1 is an external perspective view of a myoelectric measurement device 100 according to a first embodiment. FIG. 2 is a plan view of a myoelectric sensor mounting member 120 that the myoelectric measurement device 100 according to the first embodiment includes. Note that in the present embodiment, for sake of convenience, a thickness direction of the myoelectric sensor mounting member 120 is an up-down direction (Z-axis direction), a first longitudinal direction of the myoelectric sensor mounting member 120 is a front-rear direction (X-axis direction), and a second longitudinal direction of the myoelectric sensor mounting member 120 is a right-left direction (Y-axis direction).

The myoelectric measurement device 100 illustrated in FIG. 1 is a device that is mounted to an optional measurement site on a living body, and measures myoelectric signals at this measurement site. As illustrated in FIG. 1, the myoelectric measurement device 100 includes a myoelectric sensor 110 and the myoelectric sensor mounting member 120. The myoelectric measurement device 100 is configured such that the myoelectric sensor 110 is detachably mountable to the myoelectric sensor mounting member 120.

The myoelectric sensor 110 is a device that measures myoelectric signals of a measurement site of a living body. The myoelectric sensor 110 has a cuboid shape that is thin in the up-down direction (Z-axis direction). Also, the myoelectric sensor 110 has a square shape in plan view.

The myoelectric sensor 110 has a case 111. The case 111 is a container-like member made of resin, which makes up the outer shape of the myoelectric sensor 110 (i.e., the thin cuboid shape). Various types of electronic parts (e.g., an analog-to-digital converter (ADC), an integrated circuit (IC), a communication interface, a battery, and so forth) are assembled inside of the case 111, in order to realize various functions of the myoelectric sensor 110. Note that the outer shape of the case 111 is not limited to the thin cuboid shape and the square shape in plan view. For example, the outer shape of the case 111 may be a thin column shape, i.e., a circle shape in plan view.

An upper face of the case 111 is a contact face 111A that comes into contact with a measurement site of the living body. Four detection electrodes 112 are provided on the contact face 111A, protruding therefrom. Each of the four detection electrodes 112 is a metal member that detects myoelectric signals of the measurement site of the living body by coming into close contact with the skin at the measurement site of the living body. The four detection electrodes 112 are laid out in a 2×2 matrix on the contact face 111A.

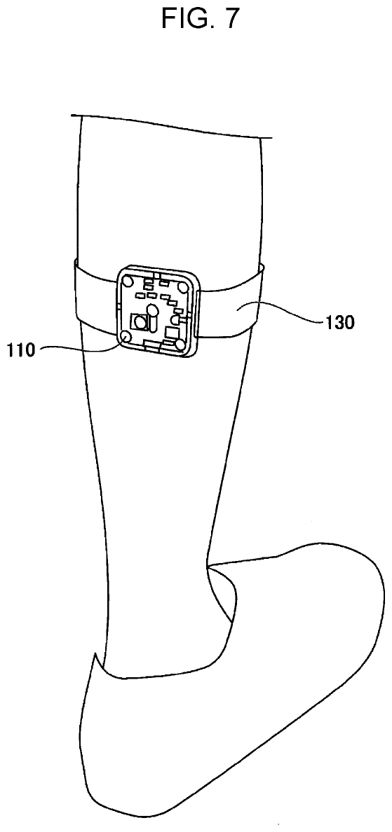

A belt mounting portion 113 may be formed on each of four sides of the contact face 111A of the case 111. Each belt mounting portion 113 has an insertion hole 113A and a support portion 113B. The insertion hole 113A is a portion in which part of the case 111 is hollowed out, from a first opening portion formed in the contact face 111A along one side of the contact face 111A and reaching a second opening portion formed on a side face of the case 111 along the one side of the contact face 111A so as to connect to the contact face 111A. The support portion 113B is a portion that is formed at a corner portion along the one side of the contact face 111A, due to the above insertion hole 113A being formed, and has a beam-like shape that spans the insertion hole 113A along this corner portion. A belt 130 that is band-like (see FIG. 7) is inserted through the insertion hole 113A, and this belt 130 is folded back with the support portion 113B as a point of support, whereby the folded-back portion of the belt 130 can be supported by the belt mounting portion 113. Accordingly, the myoelectric sensor 110 can be attached to either the myoelectric sensor mounting member 120 and the belt 130, and can be mounted at the measurement site of the living body in each of a case of using the myoelectric sensor mounting member 120 and a case of using the belt 130.

The myoelectric sensor mounting member 120 is a member for mounting the myoelectric sensor 110 to the measurement site of the living body. The myoelectric sensor mounting member 120 is formed by using an elastic material (e.g., rubber, silicone, thermoplastic polyurethane (TPU), and so forth). As illustrated in FIGS. 1 and 2, the myoelectric sensor mounting member 120 has a holding portion 122 and a mounting portion 121.

The holding portion 122 is a thin and container-like portion that opens upward (portion on negative side on Z-axis), which is the side thereof facing the measurement site of the living body. The myoelectric sensor 110 is detachably mountable to the holding portion 122. The holding portion 122 may have a recessed portion 123 that is recessed downward from an upper face thereof. The holding portion 122 may hold the myoelectric sensor 110 by the myoelectric sensor 110 being fit into the recessed portion 123 from an upper opening of the recessed portion 123.

On an inner wall face of the recessed portion 123, a plurality of groove portions 123A are formed that are continuously formed along this inner wall face. Each of the plurality of groove portions 123A has a shape that is capable of engaging corner portions of the case 111 of the myoelectric sensor 110 (substantially an isosceles right triangle shape in plan view). Accordingly, the holding portion 122 can engage each of the four corner portions of the myoelectric sensor 110 by four respective groove portions 123A, at each predetermined rotational angle of the myoelectric sensor 110. Accordingly, the holding portion 122 may be able to hold the myoelectric sensor 110 within the recessed portion 123 at each of the predetermined rotational angles.

For example, in the example illustrated in FIGS. 1 and 2, eight groove portions 123A, which are formed continuously along the inner wall face of the recessed portion 123, are formed at 45° intervals on the inner wall face of the recessed portion 123. Accordingly, the holding portion 122 may be capable of holding the myoelectric sensor 110 in the recessed portion 123 at every 45°, which is an example of a predetermined rotational angle. That is to say, the holding portion 122 is capable of holding the myoelectric sensor 110 at each of eight different rotational angles (0°, 45°, 90°, 135°, 180°, 225°, 270°, and 315′).

Note that the above “predetermined rotational angle” is not limited to 45°. For example, the holding portion 122 can hold the myoelectric sensor 110 at each of 16 different rotational angles by forming 16 groove portions 123A, which are formed continuously along the inner wall face of the recessed portion 123, at 22.5° intervals on the inner wall face thereof.

Also, both the inner wall face of the recessed portion 123 and an outer peripheral face of the case 111 of the myoelectric sensor 110 may have circle shapes in plan view, for example. Accordingly, the holding portion 122 may be able to hold the myoelectric sensor 110 at an optional rotational angle steplessly.

Note that the heightwise dimensions of the recessed portion 123 are substantially equal to the thickness dimensions of the case 111 of the myoelectric sensor 110. Accordingly, with respect to the holding portion 122, when the myoelectric sensor 110 is held in the recessed portion 123, a heightwise position of the contact face 111A of the case 111 and a heightwise position of the holding portion 122 may be made to be substantially equal. That is to say, the holding portion 122 may be able to cause each of the four detection electrodes 112 of the myoelectric sensor 110 to protrude beyond the upper face of the holding portion 122. Accordingly, the myoelectric measurement device 100 according to the present embodiment can cause each of the four detection electrodes 112 to dig into the skin at the measurement site of the living body when the upper face of the holding portion 122 and the contact face 111A of the case 111 are brought into close contact with the skin of the measurement site of the living body.

Also, as illustrated in FIG. 2, an opening portion 123B that has a circle shape in plan view is formed at the middle of an inner bottom portion of the recessed portion 123. Note that the shape of the opening portion 123B is not limited to a circle shape, and may be another shape (e.g., a square shape or the like).

As illustrated in FIGS. 1 and 2, the mounting portion 121 may have a plurality of band portions 121A provided so as to extend from the holding portion 122 in different directions from each other. In the example illustrated in FIGS. 1 and 2, the mounting portion 121 has four band portions 121A that extend from the holding portion 122 in each direction of forward (positive direction on X axis), rearward (negative direction on X axis), rightward (positive direction on Y axis), and leftward (negative direction on Y axis). Each of the plurality of band portions 121A may have, on a surface that comes into close contact with the skin of the measurement site of the living body (surface on the positive side on the Z axis), an adhesive face 121B that is capable of being applied to the skin of the measurement site of the living body. Thus, the mounting portion 121 can be securely fixed to the measurement site of the living body by the adhesive faces 121B of each of the plurality of the band portions 121A being adhered to the skin of the measurement site of the living body. Note that each of the plurality of band portions 121A is formed by using an elastic material, and accordingly can come into close contact along uneven portions of the skin of the measurement site of the living body.

### Functional Configuration of Control Unit 150

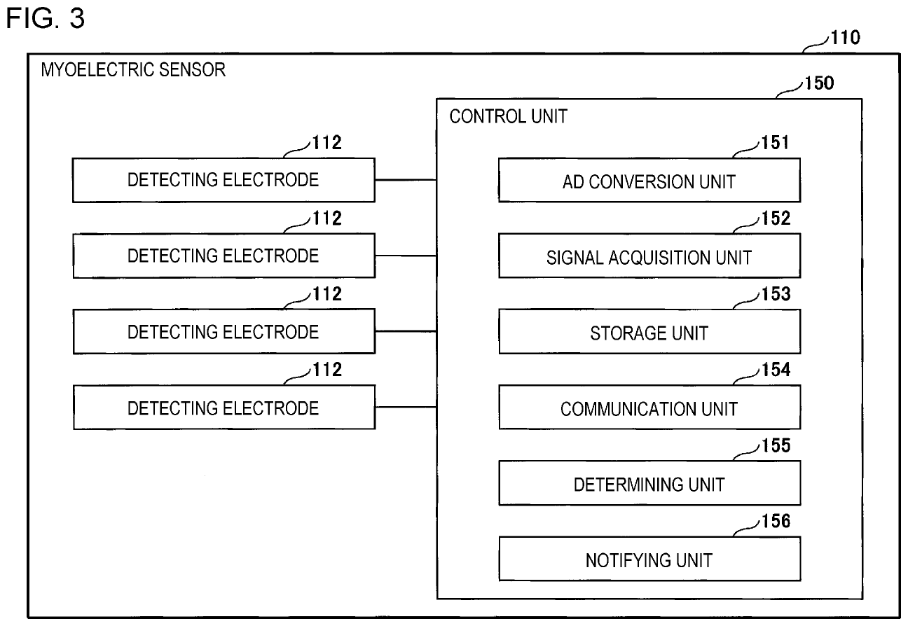

FIG. 3 is a block diagram illustrating a functional configuration of a control unit 150 that the myoelectric sensor 110 according to the first embodiment includes.

As illustrated in FIG. 3, the myoelectric sensor 110 includes the control unit 150. The control unit 150 includes, as functional portions thereof, an AD conversion unit 151, a signal acquisition unit 152, a storage unit 153, a communication unit 154, a determining unit 155, and a notifying unit 156.

The AD conversion unit 151 converts myoelectric signals (analog signals) detected by the detection electrodes 112 into digital signals. The AD conversion unit 151 is realized by an ADC that the myoelectric sensor 110 includes, for example.

The signal acquisition unit 152 acquires the myoelectric signals converted into digital signals by the AD conversion unit 151. The storage unit 153 stores the myoelectric signals acquired by the signal acquisition unit 152.

Note that the detection electrodes 112 of the myoelectric sensor 110 detect and output myoelectric signals at each predetermined detection cycle (e.g., once every second). In accordance with this, the AD conversion unit 151 converts the myoelectric signals each predetermined detection cycle. Also, the signal acquisition unit 152 acquires the myoelectric signals each predetermined detection cycle. Also, the storage unit 153 stores the myoelectric signals each predetermined detection cycle. Thus, a plurality of the myoelectric signals that are continuous in time-sequence are stored in the storage unit 153.

The communication unit 154 transmits the myoelectric signals to an external device (e.g., server device, personal computer, smartphone, or the like) via wireless communication or wired communication. Transmission of the myoelectric signals by the communication unit 154 is realized by a communication interface that the myoelectric sensor 110 includes, for example. Note that each time a myoelectric signal is acquired by the signal acquisition unit 152, the communication unit 154 may immediately transmit this myoelectric signal (e.g., real-time transmission). Alternatively, the communication unit 154 may transmit a plurality of the myoelectric signals stored in the storage unit 153 together (i.e., batch transmission), at an optional timing. For example, the communication unit 154 may perform real-time transmission of the myoelectric signals to a smartphone by Bluetooth (registered trademark) wireless communication. In this case, the smartphone can perform real-time display of measurement data of the myoelectric signals measured by the myoelectric sensor 110 on a display.

The determining unit 155 determines a suitable rotational angle for the myoelectric sensor 110 with respect to the measurement site of the living body, on the basis of results of detection of the myoelectric signals by the detection electrodes 112. A suitable rotational angle for the myoelectric sensor 110 with respect to the measurement site of the living body is a rotational angle of the myoelectric sensor 110 at which the direction of muscle fibers of a muscle at the measurement site of the living body and the direction of each of the four detection electrodes 112 are orthogonal.

For example, the determining unit 155 may determine a rotational angle of the myoelectric sensor 110 at which the intensity of myoelectric signals output by the myoelectric sensor 110 is greatest as being a suitable rotational angle of the myoelectric sensor 110.

Also, for example, the determining unit 155 may determine a rotational angle of the myoelectric sensor 110 at which the intensity of myoelectric signals output by the myoelectric sensor 110 is no less than a predetermined threshold value as being a suitable rotational angle of the myoelectric sensor 110.

The notifying unit 156 notifies a user of results of determination from the determining unit 155. For example, the notifying unit 156 may transmit the results of determination from the determining unit 155 to a smartphone by Bluetooth (registered trademark) wireless communication. In this case, the smartphone can display the results of determination by the determining unit 155 on the display. Note that the method of notification of the results of determination from the determining unit 155 is not limited to the method of displaying on a display of an external device, and in a case in which the myoelectric sensor 110 has an output device (e.g., display, speaker, light-emitting diode (LED), or the like), for example, a method of the myoelectric sensor 110 performing output from the output device may be used.

The functional portions of the control unit 150 described above (excluding the AD conversion unit 151) are realized by a central processing unit (CPU) executing a program stored in memory (e.g., read-only memory (ROM), random-access memory (RAM), or the like), in an IC included in the myoelectric sensor 110, for example.

### Example of Measurement Data of Myoelectric Signals

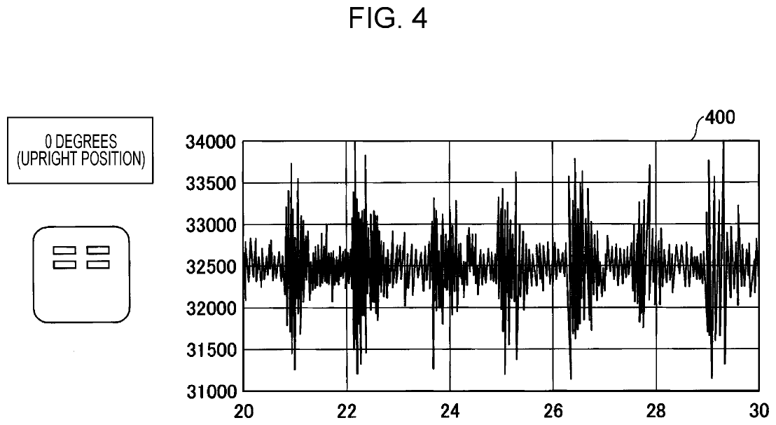

FIGS. 4 to 6 are diagrams showing an example of measurement data measured by the myoelectric sensor 110 according to the first embodiment. Each of measurement data 400 to 600 shown in FIGS. 4 to 6 is generated on the basis of a plurality of myoelectric signals detected by the myoelectric sensor 110 when the myoelectric sensor 110 is mounted on a measurement site of the living body using the myoelectric sensor mounting member 120, and indicates change in the myoelectric signals at the measurement site of the living body in time-sequence.

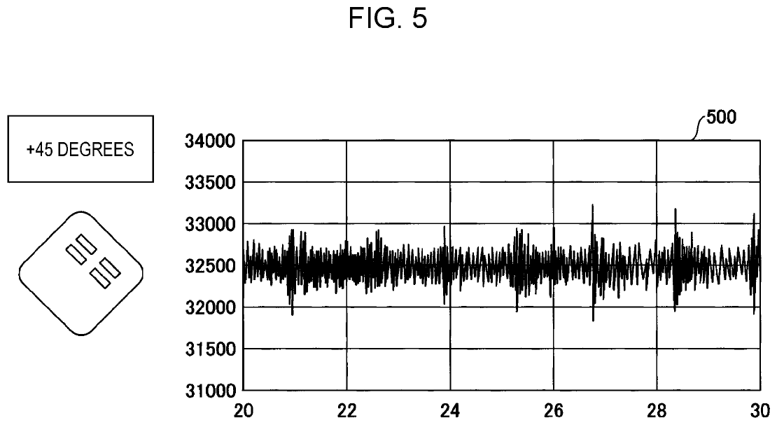

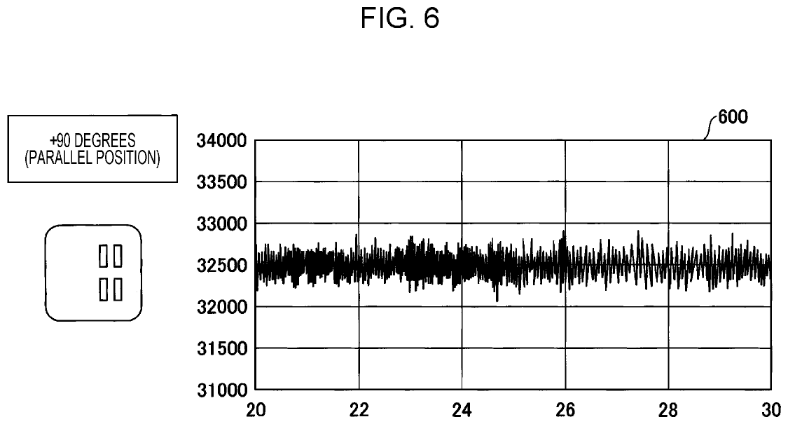

Note, however, that measurement data 400 shown in FIG. 4 is that measured by the myoelectric sensor 110 in a case in which the rotational angle of the myoelectric sensor 110 with respect to the myoelectric sensor mounting member 120 is 0°. Also, measurement data 500 shown in FIG. 5 is that measured by the myoelectric sensor 110 in a case in which the rotational angle of the myoelectric sensor 110 with respect to the myoelectric sensor mounting member 120 is 45°. Further, measurement data 600 shown in FIG. 6 is that measured by the myoelectric sensor 110 in a case in which the rotational angle of the myoelectric sensor 110 with respect to the myoelectric sensor mounting member 120 is 90°.

The intensity of the myoelectric signals is relatively great in the measurement data 400 shown in FIG. 4. This is because the direction of muscle fibers of the muscle at the measurement site of the living body and the direction of each of the four detection electrodes 112 of the myoelectric sensor 110 are orthogonal, due to the rotational angle of the myoelectric sensor 110 as to the myoelectric sensor mounting member 120 being set to 0°.

On the other hand, the intensity of the myoelectric signals is relatively small in the measurement data 500 shown in FIG. 5. Further, the intensity of the myoelectric signals is even smaller in the measurement data 600 shown in FIG. 6. This is because the direction of muscle fibers of the muscle at the measurement site of the living body and the direction of each of the four detection electrodes 112 of the myoelectric sensor 110 are not orthogonal, due to the rotational angle of the myoelectric sensor 110 as to the myoelectric sensor mounting member 120 being set to 45° and 90°.

For example, the determining unit 155 of the myoelectric sensor 110 compares the measurement data 400 to 600, and determines that the suitable rotational angle of the myoelectric sensor 110 is “0°”, since the intensity of the myoelectric signals is the greatest in the measurement data 400.

Also, for example, the determining unit 155 determines that the suitable rotational angle of the myoelectric sensor 110 is “0°” in the measurement data 400, since the intensity of the myoelectric signals is no less than a predetermined threshold value.

### Example of Mounting Myoelectric Sensor 110 by Belt 130

FIG. 7 is a diagram illustrating a mounting example of the myoelectric sensor 110 according to the first embodiment using the belt 130. In the example illustrated in FIG. 7, the belt 130 is attached to each of a pair of the belt mounting portions 113 of the myoelectric sensor 110. In this case, as illustrated in FIG. 7, wrapping the belt 130 around the measurement site of the living body (the leg in the example illustrated in FIG. 7) enables the contact face 111A of the myoelectric sensor 110 to be brought into close contact with the skin of the measurement site of the living body, and myoelectric signals at the measurement site of the living body to be detected by the four detection electrodes 112 provided on the contact face 111A. Also, in this case, rotating the orientation of the myoelectric sensor 110 by 180° along with the belt 130 enables myoelectric signals to be measured in a state in which the layout orientation of the four detection electrodes 112 is rotated by 180°. Also, attaching the belt 130 to the other pair of belt mounting portions 113 enables the orientation of the myoelectric sensor 110 to be rotated by 90° or by 270°, and myoelectric signals can be measured in a state in which the layout orientation of the four detection electrodes 112 is rotated by 90° or by 270°. That is to say, in a case of mounting the myoelectric sensor 110 to the measurement site of the living body using the belt 130, the rotational angle of the myoelectric sensor 110 can be set to any one of four different rotational angles (0°, 90°, 180°, and 270°). In this case, out of the four different rotational angles (0°, 90°, 180°, and 270°), the determining unit 155 of the myoelectric sensor 110 can determine a rotational angle at which the intensity of the myoelectric signals is greatest, or a rotational angle at which the intensity of myoelectric signals is no less than a predetermined threshold value, as being a suitable rotational angle of the myoelectric sensor 110.

As described above, with the myoelectric measurement device 100 according to the first embodiment, applying the mounting portion 121 of the myoelectric sensor mounting member 120 to the measurement site of the living body enables the contact face 111A of the myoelectric sensor 110 to be brought into close contact with the skin of the measurement site of the living body, and the myoelectric signals at the measurement site of the living body to be detected by the four detection electrodes 112 provided on the contact face 111A. Also, with the myoelectric measurement device 100 according to the first embodiment, changing the rotational angle of the myoelectric sensor 110 as to the myoelectric sensor mounting member 120 to an optional rotational angle enables the rotational angle of the myoelectric sensor 110 as to the measurement site of the living body to be changed to this optional rotational angle.

Further, the myoelectric measurement device 100 according to the first embodiment can determine a suitable rotational angle of the myoelectric sensor 110 as to the measurement site of the living body (i.e., a rotational angle at which the direction of muscle fibers of the muscle at the measurement site of the living body and the direction of each of the four detection electrodes 112 of the myoelectric sensor 110 are orthogonal) by the determining unit 155 included in the myoelectric sensor 110, on the basis of results of detection of myoelectric signals by the detection electrodes 112.

Also, the myoelectric measurement device 100 according to the first embodiment can notify the user of results of determination from the determining unit 155 (i.e., the suitable rotational angle of the myoelectric sensor 110 as to the measurement site of the living body) by the notifying unit 156 included in the myoelectric sensor 110.

Thus, according to the myoelectric measurement device 100 of the first embodiment, myoelectric signals at a wide variety of measurement sites of the living body can be detected with higher precision.

### Second Embodiment

### Configuration of Myoelectric Measurement Device 200

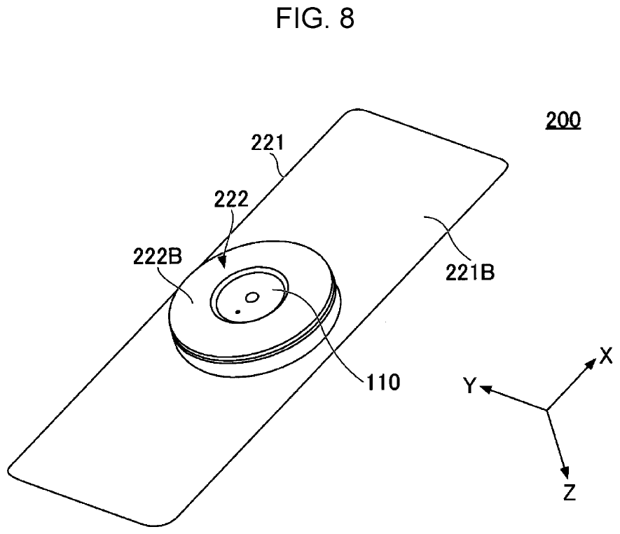

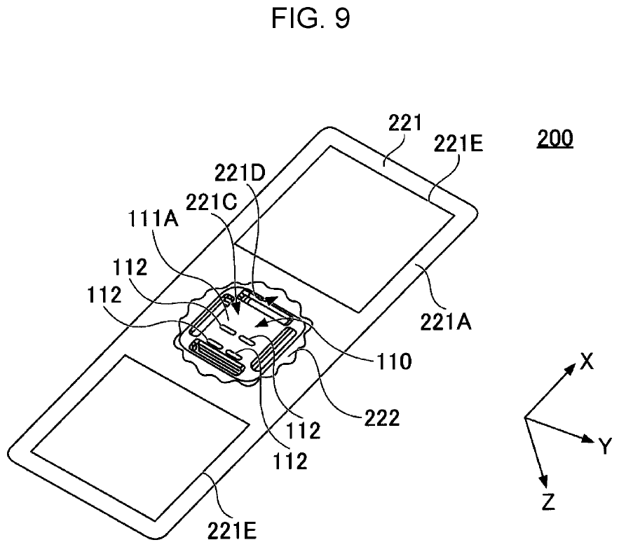

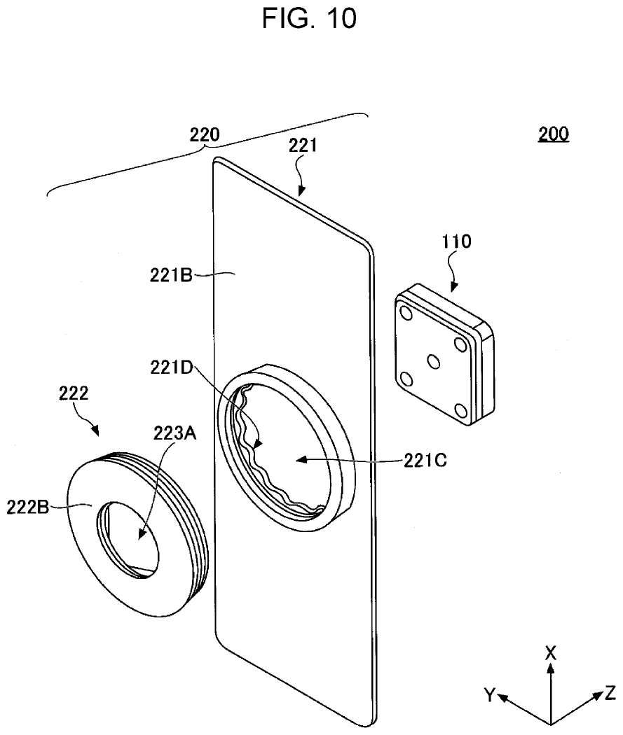

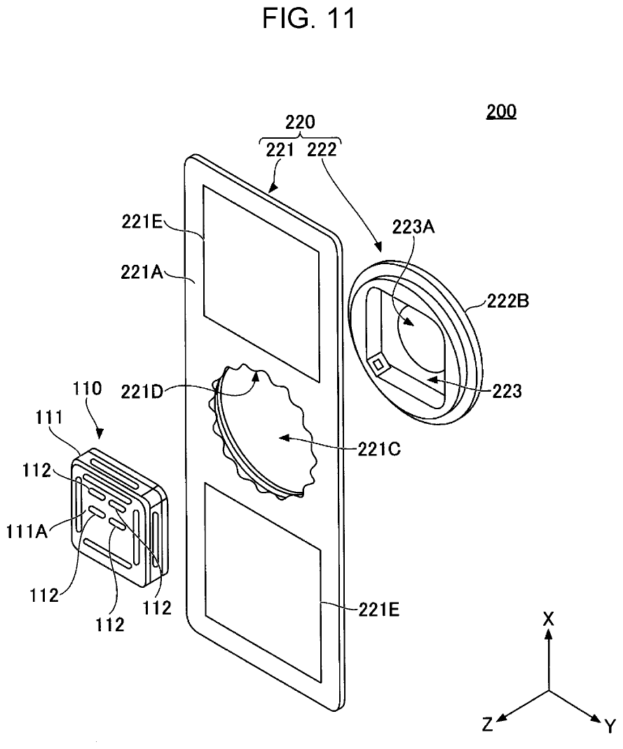

FIGS. 8 and 9 are external perspective views of a myoelectric measurement device 200 according to a second embodiment. FIGS. 10 and 11 are exploded perspective views of the myoelectric measurement device 200 according to the second embodiment. FIGS. 8 and illustrate the myoelectric measurement device 200 as viewed from the side of a measurement site of the living body. FIGS. 9 and 11 illustrate the myoelectric measurement device 200 as viewed from the side opposite to the side of the measurement site of the living body. Note that in the present embodiment, for sake of convenience, the thickness direction of a myoelectric sensor mounting member 220 is the up-down direction (Z-axis direction), a longitudinal direction of the myoelectric sensor mounting member 220 is the front-rear direction (X-axis direction), and a lateral direction of the myoelectric sensor mounting member 220 is the right-left direction (Y-axis direction).

The myoelectric measurement device 200 illustrated in FIGS. 8 to 11 is a device that is mounted to an optional measurement site of the living body, and measures myoelectric signals at this measurement site. As illustrated in FIGS. 8 to 11, the myoelectric measurement device 200 includes the myoelectric sensor 110 and the myoelectric sensor mounting member 220. The myoelectric measurement device 200 is configured such that the myoelectric sensor 110 is detachably mountable to the myoelectric sensor mounting member 220. Note that the myoelectric sensor 110 is the same as the myoelectric sensor 110 in the first embodiment.

The myoelectric sensor mounting member 220 is a member for mounting the myoelectric sensor 110 to the measurement site of the living body. As illustrated in FIGS. 8 and 9, the myoelectric sensor mounting member 220 has a holder 222 and a mounting portion 221.

The holder 222 is a thin and container-like portion of which an upper portion (portion on negative side on Z-axis), which is the side thereof facing the measurement site of the living body, is opened. The holder 222 may have a recessed portion 223 that has a shape recessed downward from an upper face thereof and that has a square shape in plan view. The holder 222 may hold the myoelectric sensor 110 by the myoelectric sensor 110 being fit into the recessed portion 223 from the upper opening of the recessed portion 223. The myoelectric sensor 110 is detachably mountable to the recessed portion 223 of the holder 222. Note that the holder 222 is a separate member from the mounting portion 221. The holder 222 is detachably mountable to the mounting portion 221. The holder 222 is formed by using a resin material. An opening portion 223A that has a circle shape in plan view is formed at the middle of an inner bottom portion of the recessed portion 223. Note that the shape of the opening portion 223A is not limited to a circle shape, and may be another shape (e.g., a square shape or the like). The holder 222 has a large-diameter portion 222B that has a larger diameter than that of an opening portion 221C of the mounting portion 221, on a face of the mounting portion 221 that is a non-contact face 221B side. Also, the recessed portion 223 is not limited to having the square shape in plan view. That is to say, in a case in which the myoelectric sensor 110 has another shape in plan view (e.g., circle shape, rectangle shape, or the like), the recessed portion 223 may have that other shape (e.g., circle shape, rectangle shape, or the like), such that the myoelectric sensor 110 can be fit therein.

The mounting portion 221 is a member for mounting the myoelectric sensor 110 to the measurement site of the living body. The mounting portion 221 is a band-like member of which the front-rear direction (X-axis direction) is the longitudinal direction, and the right-left direction (Y-axis direction) is the lateral direction. The mounting portion 221 is formed by using an elastic material (e.g., rubber, silicone, TPU, and so forth).

One face (face on positive side on Z axis) of the mounting portion 221 is a contact face 221A that comes into contact with the skin of the measurement site of the living body. The other face (face on negative side on Z axis) of the mounting portion 221 is the non-contact face 221B that does not come into contact with the skin of the measurement site of the living body.

The mounting portion 221 has, at the middle portion thereof, the opening portion 221C that has a circle shape. The holder 222 is fit into the opening portion 221C from the non-contact face 221B side of the mounting portion 221. Accordingly, the opening portion 221C rotatably supports the holder 222. That is to say, in the present embodiment, the opening portion 221C and the holder 222 make up a “holding portion capable of holding the myoelectric sensor at an optional rotational angle”.

With respect to the holder 222, the large-diameter portion 222B thereof may protrude beyond the non-contact face 221B of the mounting portion 221 in a state of being fit into the opening portion 221C, and accordingly rotational operation of the mounting portion 221 by the large-diameter portion 222B is enabled from the non-contact face 221B side. Also, in a state in which the holder 222 is fit into the opening portion 221C, the myoelectric sensor 110 is fit into the recessed portion 223 of the holder 222 from the contact face 221A side of the mounting portion 221. Accordingly, the myoelectric sensor 110 is capable of rotating along with the holder 222 in the opening portion 221C.

Note that in a state in which the holder 222 is fit into the opening portion 221C a height position of the contact face 111A of the myoelectric sensor 110 that is held by the holder 222 and a height position of the contact face 221A of the mounting portion 221 may be equal to each other. In addition, each of the four detection electrodes 112 of the myoelectric sensor 110 may be provided so as to protrude beyond the contact face 111A. Accordingly, when the myoelectric measurement device 200 according to the present embodiment is mounted to the measurement site of the living body, the contact face 111A of the myoelectric sensor 110 can be brought into close contact with the skin of the measurement site of the living body, and the each of the four detection electrodes 112 of the myoelectric sensor 110 can be made to dig into the skin of the measurement site of the living body.

On an inner wall face of the contact face 221A side of the opening portion 221C, a plurality of groove portions 221D are formed that are continuously formed along this inner wall face. Each of the plurality of groove portions 221D has a shape that is capable of engaging corner portions of the case 111 of the myoelectric sensor 110 (substantially an isosceles right triangle shape in plan view). Accordingly, the mounting portion 221 can engage each of the four corner portions of the myoelectric sensor 110 by four respective groove portions 221D, at each predetermined rotational angle of the holder 222 and the myoelectric sensor 110. Accordingly, the mounting portion 221 may be able to hold the holder 222 and the myoelectric sensor 110 within the opening portion 221C at each of predetermined rotational angles.

Note that the opening portion 221C does not need to have the plurality of groove portions 221D. In this case, the mounting portion 221 can hold the holder 222 and the myoelectric sensor 110 at an optional rotational angle, steplessly.

The contact face 221A of the mounting portion 221 has an adhesive face 221E that is capable of being applied to the skin of the measurement site of the living body, on each of a band-like portion that extends forward (positive direction on X axis) from the opening portion 221C and a band-like portion that extends rearward (negative direction on X axis) from the opening portion 221C. Thus, the mounting portion 221 can be securely fixed to the measurement site of the living body, by the adhesive faces 221E being adhered to the skin of the measurement site of the living body. Note that the mounting portion 221 is formed by using an elastic material, and accordingly can come into close contact along uneven portions of the skin of the measurement site of the living body.

With the myoelectric measurement device 200 according to the second embodiment, wrapping the mounting portion 221 onto a measurement site of the living body (e.g., leg, arm, or the like) enables the contact face 111A of the myoelectric sensor 110 to be brought into close contact with the skin of the measurement site of the living body, and the myoelectric signals at the measurement site of the living body to be detected by the four detection electrodes 112 provided on the contact face 111A.

Also, in a state in which the myoelectric measurement device 200 according to the second embodiment is mounted to the measurement site of the living body, the myoelectric sensor 110 held by the holder 222 can be rotated to an optional rotational angle by rotating the holder 222.

Further, the myoelectric measurement device 200 according to the second embodiment can determine a suitable rotational angle for the myoelectric sensor 110 as to the measurement site of the living body (i.e., a rotational angle at which the direction of muscle fibers of the muscle at the measurement site of the living body and the direction of each of the four detection electrodes 112 of the myoelectric sensor 110 are orthogonal) by the determining unit 155 included in the myoelectric sensor 110, on the basis of results of detection of myoelectric signals by the detection electrodes 112.

Also, the myoelectric measurement device 200 according to the second embodiment can notify the user of results of determination from the determining unit 155 (i.e., the suitable rotational angle of the myoelectric sensor 110 as to the measurement site of the living body) by the notifying unit 156 included in the myoelectric sensor 110.

Thus, according to the myoelectric measurement device 200 of the second embodiment, myoelectric signals at a wide variety of measurement sites of the living body can be detected with higher precision.

Detailed description of the present disclosure has been made above by way of embodiments, but the present disclosure is not limited to these embodiments, and various modifications and alterations can be made without departing from the spirit and scope of the present disclosure, or the inventions set forth in the Claims.

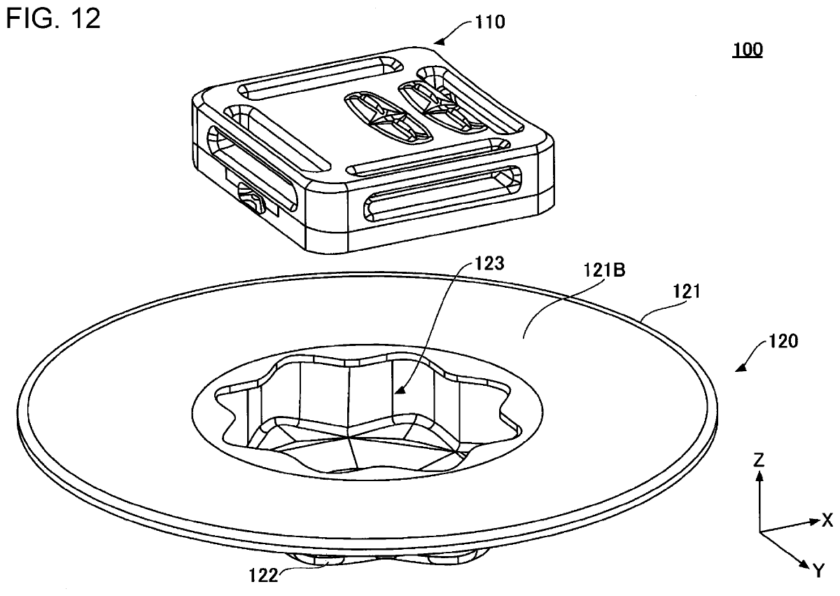

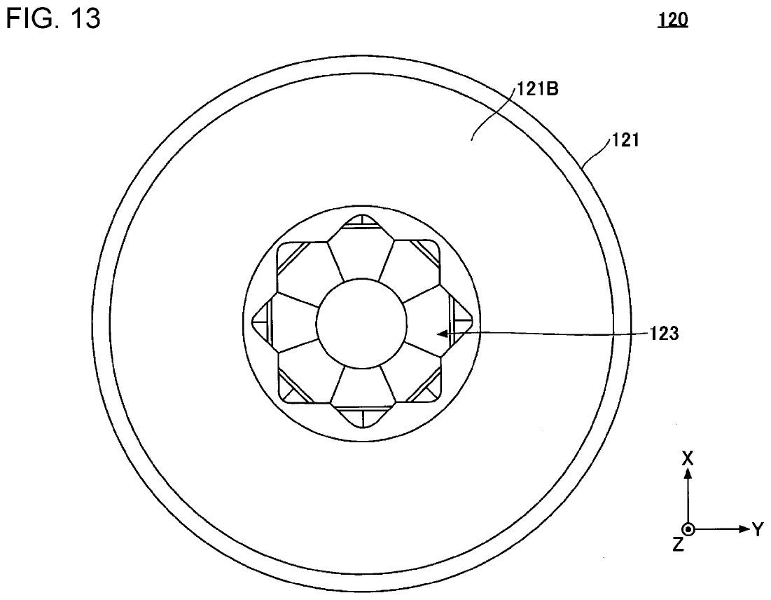

For example, FIG. 12 is an external perspective view illustrating a modification of the myoelectric measurement device 100 according to the first embodiment. FIG. 13 is a plan view illustrating a modification of the myoelectric sensor mounting member 120 that the myoelectric measurement device 100 according to the first embodiment includes. As illustrated in FIGS. 12 and 13, for example, the shape of the mounting portion 121 included in the myoelectric sensor mounting member 120 may be annular or radial in plan view. In accordance therewith, the adhesive face 121B may be annular or radial in plan view, as illustrated in FIGS. 12 and 13.

What is claimed is:

1. A myoelectric sensor mounting member comprising:
a mounting portion for mounting to a measurement site of a living body; and,
a holding portion capable of holding a myoelectric sensor at an optional rotational angle, the myoelectric sensor comprising a detection electrode on a contact face for contacting the measurement site;
wherein an orientation of the detection electrode as to the measurement site can be changed by changing the rotational angle at which the holding portion holds the myoelectric sensor.

2. The myoelectric sensor mounting member according to claim 1, wherein the holding portion comprises:
a recessed portion for accommodating the myoelectric sensor; and,
a plurality of groove portions continuously formed along an inner wall face of the recessed portion;
wherein the myoelectric sensor can be held at each of predetermined rotational angles by the myoelectric sensor engaging a part of the groove portions at each of the predetermined rotational angles in the recessed portion.

3. The myoelectric sensor mounting member according to claim 1, wherein:
the mounting portion comprises a plurality of band portions provided so as to extend from the holding portion in different directions from each other.

4. The myoelectric sensor mounting member according to claim 3, wherein:
each of the plurality of band portions has an adhesive face capable of being applied to the living body.

5. The myoelectric sensor mounting member according to claim 1, wherein:
the mounting portion has an annular shape in plan view.

6. The myoelectric sensor mounting member according to claim 5, wherein:
the mounting portion has an adhesive face capable of being applied to the living body.

7. The myoelectric sensor mounting member according to claim 1, wherein the holding portion comprises:
an opening portion; and,
a holder that holds the myoelectric sensor, and that is capable of rotating in the opening portion;
wherein a large-diameter portion of the holder protrudes beyond a non-contact face of the mounting portion.

8. The myoelectric sensor mounting member according to claim 7, wherein:
the holder is capable of rotating at each of predetermined rotational angles in the opening portion.

9. The myoelectric sensor mounting member according to claim 7, wherein:
the holder is capable of rotating steplessly in the opening portion.

10. A myoelectric measurement device comprising:
a myoelectric sensor comprising a detection electrode on a contact face for contacting a measurement site of a living body;
a mounting portion for mounting to the measurement site; and,
a holding portion capable of holding the myoelectric sensor at an optional rotational angle;
wherein an orientation of the detection electrode as to the measurement site can be changed by changing the rotational angle at which the holding portion holds the myoelectric sensor.

11. The myoelectric measurement device according to claim 10, wherein the myoelectric sensor comprises:
a belt mounting portion to which a belt can be attached for mounting the myoelectric sensor to the measurement site.

12. The myoelectric measurement device according to claim 10, wherein the myoelectric sensor comprises:
a processor configured to determine a suitable rotational angle for the myoelectric sensor as to the measurement site, on the basis of a result of detection in which intensity of the myoelectric signal from the detection electrode is greatest, or the intensity thereof is no less than a predetermined threshold value.

13. The myoelectric measurement device according to claim 10, wherein:
a height position of the contact face of the myoelectric sensor and a height position of a contact face of the mounting portion are equal to each other, and the detection electrode of the myoelectric sensor is provided so as to protrude beyond the contact face of the myoelectric sensor.

14. A myoelectric measurement device comprising:
a myoelectric sensor comprising a detection electrode on a contact face for contacting a measurement site of a living body;
a mounting portion for mounting to the measurement site;
a holding portion capable of holding the myoelectric sensor at an optional rotational angle, wherein an orientation of the detection electrode as to the measurement site can be changed by changing the rotational angle at which the holding portion holds the myoelectric sensor; and,
a processor configured to determine a suitable rotational angle for the myoelectric sensor as to the measurement site, on the basis of a result of detection in which intensity of the myoelectric signal from the detection electrode is greatest, or the intensity thereof is no less than a predetermined threshold value.

15. The myoelectric measurement device according to claim 14, wherein the holding portion comprises:
a recessed portion for accommodating the myoelectric sensor; and,
a plurality of groove portions continuously formed along an inner wall face of the recessed portion;
wherein the myoelectric sensor can be held at each of predetermined rotational angles by the myoelectric sensor engaging a part of the groove portions at each of the predetermined rotational angles in the recessed portion.

16. The myoelectric measurement device according to claim 14, wherein the holding portion comprises:
an opening portion; and,
a holder that holds the myoelectric sensor, and that is capable of rotating in the opening portion;
wherein a large-diameter portion of the holder protrudes beyond a non-contact face of the mounting portion.

17. The myoelectric measurement device according to claim 16, wherein:
the holder is capable of rotating at each of predetermined rotational angles in the opening portion.

18. The myoelectric measurement device according to claim 16, wherein:
the holder is capable of rotating steplessly in the opening portion.
